(19) United States
(12) Patent Application Publication
(10) Pub. No.: US 2009/0234012 A1
(43) Pub. Date: Sep. 17, 2009

(54) ADMINISTRATION OF DOPA PRECURSORS WITH SOURCES OF DOPA TO EFFECTUATE OPTIMAL CATECHOLAMINE NEUROTRANSMITTER OUTCOMES

(75) Inventor: Martin C. Hinz, Duluth, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Martin C. Hinz, Duluth, MN (US)

(21) Appl. No.: 12/400,291

(22) Filed: Mar. 9, 2009

Related U.S. Application Data
(63) Continuation of application No. 11/759,732, filed on Jun. 7, 2007, now abandoned.
Application No. 11/759,732 is a continuation-in-part of application No. 12/058,338, filed on Mar. 28, 2008.
Application No. 12/058,338 is a continuation of application No. 10/394,597, filed on Mar. 21, 2003, now abandoned.
Application No. 10/394,597 is a continuation-in-part of application No. 11/282,965, filed on Nov. 18, 2005.
Application No. 11/282,965 is a continuation-in-part of application No. 10/785,158, filed on Feb. 23, 2004, now abandoned.
(60) Provisional application No. 60/811,844, filed on Jun. 8, 2006.
(60) Provisional application No. 60/366,983, filed on Mar. 21, 2002.
(60) Provisional application No. 60/449,229, filed on Feb. 21, 2003.

(51) Int. Cl. A61K 31/197

(52) U.S. Cl. 514/567

(57) ABSTRACT

A method of treating neurotransmitter dysfunction in a patient by optimizing catecholamine levels by administration of L-3,4-dihydroxyphenylalanine (L-Dopa or Dopa) precursors in combination with a source of L-Dopa. The dopa precursor is preferably administered in such quantities such that the amount of dopa from the dopa precursors does not fluctuate and affect outcomes in the synthesis of dopamine from dopa administration. The dopa precursor source is preferably tyrosine, but may alternatively be phenylalanine, N-acetyl-tyrosine, any active isomer thereof, or any other dopa precursor. The source of L-Dopa may include any natural or synthetic source, including, but not limited to, Mucuna pruriens.

### RELATED APPLICATION

This application is claims the benefit of U.S. Provisional Application No. 60/811,844 filed Jun. 8, 2006, hereby fully incorporated herein by reference.

### FIELD OF THE INVENTION

The present invention relates, generally, to biomedical technology. More particularly, the invention relates to a technology for optimizing control of catecholamine levels by administration of L-3,4-dihydroxyphenylalanine (L-Dopa or Dopa) precursors in combination with a source of L-Dopa. Most particularly, the invention relates to safe, effective compositions, methods, therapies and techniques for managing catecholamine levels and levels of substances where catecholamines are a precursor in subjects with a serotonin and catecholamine neurotransmitter system in order to optimize individual and group outcomes in the treatment of neurotransmitter dysfunction and dysfunction of systems regulated or controlled by the serotonin and/or catecholamine systems. The compositions, methods and techniques of the invention have broad applicability with respect to neurotransmitter dysfunction, including disease. The compositions, methods, and techniques may also be useful in other fields.

### BACKGROUND OF THE INVENTION

As previously taught in U.S. patent application Ser. No. 10/785,158 and U.S. patent application Ser. No. 10/394,597, which are herein incorporated by reference, there is a correlation between the master neurotransmitters such as serotonin and/or catecholamine systems (dopamine, norepinephrine, and epinephrine) and resolution of disease symptoms. Neurotransmitter dysfunction associated with the catecholamine and/or serotonin system may include, but is not limited to, depression, anxiety, panic attacks, migraine headache, obesity, bulimia, anorexia, premenstrual syndrome, menopause, insomnia, hyperactivity, attention deficit disorder, impulsivity, obsessionality, aggression, inappropriate anger, psychotic illness, obsessive compulsive disorder, fibromyalgia, chronic fatigue syndrome, chronic pain states, adrenal fatigue, attention deficit hyperactivity disorder, Parkinsonism, and states of decreased cognitive function such as dementia and Alzheimer's disease.

It is known in the serotonin synthesis pathway, which is shown below, that Serotonin is synthesized from L-tryptophan and L-5-hydroxytryptophan (5-HTP) in the body (peripheral) and the brain (central). Vitamin B3 is a cofactor in the synthesis of 5-HTP from tryptophan. Vitamin B6 and Vitamin C are cofactors in the synthesis of serotonin from 5-HTP. Serotonin synthesis is regulated by the “serotonin-tryptophan hydroxylase feedback loop.” As increasing amounts of serotonin are synthesized, it binds to and shuts down the tryptophan hydroxylase enzyme, effectively regulating and limiting the amount of serotonin that can be synthesized in the body. With 5-HTP administration, there is no regulation of the synthesis of serotonin.

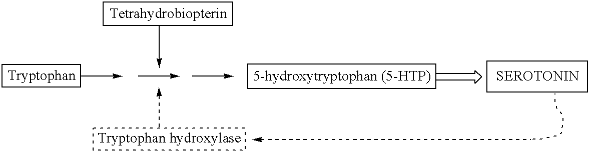

It is also known that in the catecholamine synthesis pathway the rate of dopamine synthesis, and subsequent products of such synthesis where dopamine acts as a precursor, is controlled by the “norepinephrine/tyrosine hydroxylase feed back loop,” which is shown below. Epinephrine also inhibits tyrosine hydroxylase.

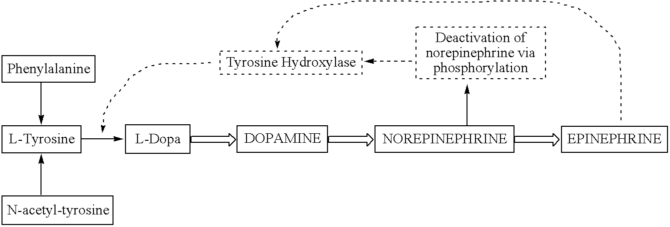

The catecholamines are synthesized in the body (peripheral) and in the brain (central) from either the amino acid precursors L-tyrosine or L-dopa. L-phenylalanine and N-acetyl-tyrosine are also precursors of the catecholamines further up the catecholamine synthesis pathway, which are further regulated be chemical feedback loops (not shown).

Prior patent applications by the Applicant have also taught that the central nervous system neurotransmitter levels in the brain can be increased by administration of amino acid precursors of the serotonin and catecholamine neurotransmitters. Such amino acid precursors include: tryptophan, 5-hydroxytryptophan, tryosine, and dopa, which cross the blood brain barrier and are then synthesized in the central nervous system into the respective neurotransmitters. The amino acid precursors phenylalanine and N-acetyl-tyrosine may also be ultimately synthesized into dopamine, but they are further down the synthesis pathway and are more heavily regulated by feedback loops. Also, they can be affected by other synthesis needs using the precursor involved or its products of synthesis.

As shown in the Catecholamine Synthesis Pathway above, norepinephrine is synthesized without feed back regulation from dopamine. Norepinephrine can then bind to one of the four ligand legs of the tyrosine hydroxylase enzyme rendering it less active. When all four binding sites of the tyrosine hydroxylase enzyme are occupied by norepinephrine, complete shut down of the enzyme's ability to catalyze synthesis of dopa from tyrosine occurs. Literature teaches that when four molecules of norepinephrine bind to the four ligand legs of tyrosine hydroxylase the tyrosine hydroxylase is rendered inactive and in a state where it can no longer effectuate the synthesis of dopamine from tyrosine. Applicant's research and original work, however, leads to the observation that the shutting down of the tyrosine hydroxylase enzyme by norepinephrine is not an absolute or complete process.

### SUMMARY OF THE INVENTION

In a first aspect, the invention pertains to a method of administering a precursor of dopa in combination with a source of dopa to stabilize catecholamine neurotransmitter levels and effectuate optimal outcomes in a subject. In some embodiments, the method can include tyrosine as the precursor of dopa. In some embodiments, the method can include phenylalanine as the precursor of dopa. In some embodiments, the method can include N-acetyl-tyrosine as the precursor of dopa. In some embodiments, the method can include a combination of tyrosine, phenylalanine, and/or N-acetyl-tyrosine as the precursor of dopa.

In a further aspect, the invention pertains to a method of stabilizing catecholamine neurotransmitter levels of a subject within a desired range by establishing an underlying stream of dopa being synthesized through administration of a precursor of dopa in combination with a direct source of dopa. In some embodiments, the method can include a combination of tyrosine, phenylalanine, and/or N-acetyl-tyrosine as the precursor of dopa that provides the underlying stream of dopa. In some embodiments, the direct source of dopa can be a natural or synthetic source of dopa, such as a Mucuna pruriens extract standardized to a percentage of dopa content.

In a further aspect, desired neurotransmitter levels of dopamine, epinephrine, and norepinephrine in subjects can be achieved by administering a proper base of dopa precursors after the serotonin neurotransmitter levels are stabilized with a combination of serotonin precursor and dopa precursor. In some embodiments, the proper base of dopa precursors includes an increase in the amount of dopa precursor after the serotonin neurotransmitter levels are stabilized in the subject. In some embodiments, the proper base of dopa precursor is administered in combination with dopa.

### BRIEF DESCRIPTION OF THE DRAWINGS

FIG. 1 is an illustration of the catecholamine neurotransmitters showing that tyrosine hydroxylase is the rate limiting step in dopamine synthesis, and that since norepinephrine and epinephrine inhibit tyrosine hydroxylase, pharmacologically modulating one neurotransmitter may affect levels of other neurotransmitters.

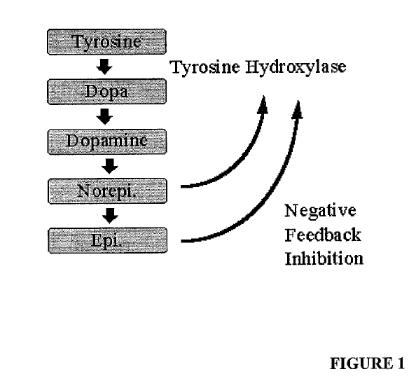

### DETAILED DESCRIPTION OF THE INVENTION

The embodiment of the invention described is intended to be illustrative and not to be exhaustive or limit the invention to the exact forms disclosed. The embodiments are chosen and described so that persons skilled in the art will be able to understand the invention and the manner and process of making and using it.

The present invention involves the use of a precursors of dopa such as but not limited to phenylalanine, N-acetyltyrosine or tyrosine with dopa to stabilize and give more predictable outcomes in the administration of dopa as a precursor in the synthesis of dopamine, with or without laboratory assay of the neurotransmitter dopamine and/or norepinephrine and/or epinephrine, or other substances where dopamine may be a precursor.

The need for the present invention is established through the review of laboratory catecholamines (dopamine, norepinephrine, and epinephrine) assays where dopamine precursors alone were used to affect change in catecholamine levels (dopamine, norepinephrine, epinephrine). Analysis of laboratory results leading up to this invention, from over 5,000 subjects, shows that administration of individual dopamine precursors such as tyrosine, N-acetyltyrosine, phenylalanine, or dopa results in significant problems with controlling dopamine levels with the use of the precursor. In turn this leads to problems in controlling the products of synthesis where dopamine is a precursor such as norepinephrine and epinephrine. In general these problems extend to all products of synthesis where dopamine is a precursor. It is the teaching of the present invention that administration of a single dopamine precursor can lead to laboratory results that are difficult to control and which fluctuate wildly in some subjects. These problems are counterproductive especially when the goal of precursor administration is to achieve dopamine levels in a desired range. This fluctuation of laboratory assayed dopamine levels also extends to all products of synthesis where dopamine is a precursor, including, but not limited to, metabolites and other neurotransmitters where dopamine is a precursor.

The present invention alleviates the foregoing problems by administration a dopa precursor, such as, but not limited to, phenylalanine, N-acetyltyrosine, or tyrosine, in combination with dopa to effectuate desired laboratory assay results and/or clinical outcomes. These desired laboratory assay results and/or clinical outcomes are:
1. Much more predictable;
2. More stable;
3. Less prone to fluctuation;
4. More stable with regards to outcomes with products synthesized where dopamine is a precursor;
5. More stable outcomes in clinic applications where dopamine is involved;
6. Able to expedite greatly the amount of time and testing needed to establish dopamine levels and products synthesized where dopamine is a precursor in a desired range; and/or
7. Able to markedly decrease the amount of administered dopa needed to achieve desired results in applications where dopa administration is desirable.

As discussed above, it is known that the rate of dopamine synthesis, and subsequent products of synthesis where dopamine acts as a precursor, is controlled by the “norepinephrine/tyrosine hydroxylase feed back loop.” Norepinephrine is synthesized without feed back regulation from dopamine. Norepinephrine can then bind to one of the four ligand legs of the tyrosine hydroxylase enzyme rendering it inactive. When all four binding sites are occupied by norepinephrine, complete shut down of the enzyme's ability to catalyze synthesis of dopa from tyrosine occurs. Literature teaches that when four molecules of norepinephrine bind to the four ligand legs of tyrosine hydroxylase the tyrosine hydroxylase is rendered inactive and in a state where it can no longer effectuate the synthesis of dopamine from tyrosine.

Research and collected data leading up to the present invention supports the observations and conclusion that the shutting down of the tyrosine hydroxylase enzyme by norepinephrine is not an absolute or complete process. Instead, even when large amounts of dopa are administered, there continues to be two sources of dopa being synthesized into dopamine. One source is the direct administration of dopa. The second source is the dopa that continues to be synthesized by the tyrosine hydroxylase enzyme from dopa precursors. This second source continues to play a significant role as a precursor of dopamine even in the face of extremely large amounts of dopa being administered. Further, it is apparent that in dopa administration in all life forms containing a dopamine neurotransmitter system there is an underlying stream of dopa being synthesized from tyrosine no matter what the dosing level of dopa.

Administration of a single precursor of dopamine such as but not limited to tyrosine, N-acetyltyrosine, phenylalanine, or dopa does not allow for optimal control of dopamine. For example, in administration of dopa precursors such as but not limited to tyrosine, N-acetyltyrosine, or phenylalanine the norepinephrine/tyrosine hydroxylase feed back loop does limit the maximum amount of dopamine that may be synthesized. But levels can be increased significantly under this maximum with administration of these single precursors alone.

Administration of the single dopamine precursor dopa is not subject to the norepinephrine/tyrosine hydroxylase feed back loop and has the ability to raise dopamine levels infinitely high if infinitely high levels of the precursor are administered. However, the observed problem is that serial laboratory assays of the results of administration of only dopa reveals that dopamine levels fluctuate wildly at times causing the ability to obtain stable dopamine levels to be almost impossible in some subjects.

It is the teaching of the invention that for optimal control and results of dopamine as well as subsequent synthesis where dopamine is a precursor, there must be a use of the combination of dopa with a precursor of dopa in proper levels. In order to obtain optimal results in the synthesis of dopamine with administration of dopa, the underlying stream of dopa being synthesized from precursors of dopa must be addressed through administration of a dopa precursor in combination with dopa.

If the underlying stream of dopa synthesized from dopa precursors in dopa administration is not properly addressed through adequate administration of proper levels of dopa precursors with the dopa, dopamine synthesized from dopa administered tends to fluctuate widely as the underlying stream of dopa from dopa precursors fluctuates. When proper levels of dopa precursors are administered in combination with the dopa so that the underlying stream of dopa from dopa precursors does not fluctuate and affect outcomes in the synthesis of dopamine from dopa, stable levels of dopamine and other catecholamines may be achieved.

The present invention teaches that for optimal control of dopamine levels a “dopa precursor base” must be used in combination with administration of dopa. This is a consideration in dopa administered in any dosing range. With regards to the dosing range of dopamine precursors needed in dopa administration, selected dosing of some precursors are as follows:
1. Tyrosine 50 mg to 14,000 mg per day
2. N-acetyltyrosine 50 mg to 14,000 mg per day
3. Phenylalanine 50 mg to 14,000 mg per day

In a preferred embodiment, the dosing range of the dopa precursor may be in the range of about 750 to 9,000 mg per day. The dosing range of the dopa may be in a range of about 12 mg to 4800 mg per day. If dopa is administered without a proper “dopa precursor base” being put in place, the dopamine outcomes of synthesis as displayed in laboratory assay and/or clinical results may not stabilize to desired levels and fluctuate wildly at times. In an embodiment, it is desirable to stabilize the dopamine levels within a range of about 20 percent of the previously assayed level. This level of variability is independent of any variability attributable to the laboratory testing methodology.

The discussion above relates to an adult human. The present invention may also be applied to any life form containing a dopamine system where dopamine is synthesized from a precursor.

In general, pediatric dosing is defined as a human 16 years of age or less although subjects as young as 10 years old with adult dosing needs have been observed while subjects as old at 20 years old appear to have pediatric dosing needs. In general the pediatric dosing starting point is one half that of adult dosing.

In other life forms, dosing is adjusted on a milligram per kilogram basis using 50 kilograms as a reference point for the full dose.

Laboratory assay of neurotransmitters of the serotonin and catecholamine systems can be carried out by assay of serum, saliva, urine, or any other method which accurately reflects the neurotransmitter levels of the serotonin and catecholamine systems. The advantages and disadvantages of assays of serum, saliva, and urine to accurately reflect the neurotransmitter levels in the serotonin and catecholamine systems was previously discussed in U.S. patent application Ser. No. 10/785,158 and U.S. patent application Ser. No. 11/282,965, which are both hereby incorporated by reference.

The method opted for as the method of choice in assay of neurotransmitter levels is urinary neurotransmitter testing. This assay is not a completely straight forward assay and must be preformed with adherence to the following considerations. In reporting urinary assay results consideration must be made to compensate for dilution of the urine (specific gravity variance). Simply assaying the neurotransmitters in a given urine sample will not give results of desired meaning due to variance in specific gravity from sample to sample. One method to compensate for variance in specific gravity is to report the results as a neurotransmitter to creatinine ratio. The preferred method is reporting results as micrograms of neurotransmitter per gram of creatinine in the urine. In utilizing urinary laboratory assay of neurotransmitters the problem of minute-to-minute spikes in the neurotransmitter levels is overcome and the results reported are an average of the neurotransmitters levels in the urine since the bladder was last emptied (generally 2 to 3 hours earlier). Other considerations of urinary neurotransmitter assay include, but are not limited to, the urine should not be collected first thing in the morning unless you are assaying neurotransmitter levels during the night. Contrary to the usual method for collection of urine for neurotransmitter assay where a pathologic diagnosis of pheochromocytoma, a serotonin secreting tumor, and the like is being made, the urine used in assay of neurotransmitters in support of amino acid therapy of the serotonin and catecholamine systems should be collected late in the day (preferably 5 to 6 hours before bed time) when the neurotransmitter levels are at their lowest. In the case where pathologic diagnosis is being made or in lab testing to assist in establishing neurotransmitter levels in the optimal range throughout the day, or to gauge situations of neurotransmitter overload and toxicity it is desirable to collect urine in the AM when neurotransmitter levels are at their highest so as to demonstrate peak levels. Urinary assay of neurotransmitters in support of amino acid therapy of the serotonin and catecholamine systems should be collected at or near the low point, 5 or 6 hours before bed time, to insure that a neurotransmitter assay is obtained in an effort to ensure that neurotransmitter levels do not drop below levels needed to keep the system free of disease symptoms (a therapeutic range), although collections at other times of the day may yield meaningful results which are less than optimal.

The primary application of laboratory assay of neurotransmitters of the serotonin and catecholamine systems is to assist in establishing therapeutic levels of neurotransmitters, which correlate with the resolution of disease symptoms. The first step in laboratory testing is to define a reference range via statistical analysis of the population as is standard practice for laboratories. For example, one respective laboratory reference range of serotonin may be defined as 100 to 250 micrograms of serotonin per gram of creatinine. It is recognized that many people with urinary neurotransmitter assay values inside of the reference range are suffering from neurotransmitter dysfunction related illness and the only way to provide effective relief of symptoms is to establish neurotransmitter levels that are higher than the reference range in what is known as the therapeutic range. The Parkinson's disease model illustrates very well why higher than normal levels are needed in many subjects not just in Parkinsonism. But still there is a subgroup of people who have no symptoms of neurotransmitter dysfunction and are functioning at a very high level. In studying this group of subjects, an optimal range was defined inside the reference range.

The following laboratory value numbers are for the specific laboratory used in the research of this invention. Due to variability in assay techniques between laboratories actual values may legitimately vary from laboratory to laboratory.

“REFERENCE RANGES” are the ranges set by the individual laboratory from statistical analysis of a population of subjects based on defining the mean and standard deviation. The typical reference range is the value found in calculating two standard deviations above and below the mean. The reference range reported by each laboratory may also be unique depending on the methodology of the assay being used. An exemplary embodiment of the reference range established by a first laboratory is as follows:

Serotonin=100 to 250 micrograms of neurotransmitter per gram of creatinine.

Dopamine=100 to 250 micrograms of neurotransmitter per gram of creatinine.

Norepinephrine=25 to 75 micrograms of neurotransmitter per gram of creatinine.

Epinephrine=5 to 13 micrograms of neurotransmitter per gram of creatinine.

Another exemplary embodiment of the reference range established by a second laboratory is as follows:

Serotonin=48.9 to 194.9 micrograms of neurotransmitter per gram of creatinine.

Dopamine=40.0 to 390.0 micrograms of neurotransmitter per gram of creatinine.

Norepinephrine=7.0 to 65.0 micrograms of neurotransmitter per gram of creatinine.

Epinephrine=2.0 to 16.0 micrograms of neurotransmitter per gram of creatinine.

OPTIMAL RANGES are defined as a narrow range within the reference range where subjects with no symptoms of neurotransmitter dysfunction appear to be functioning optimally based on group observations. The optimal ranges for the neurotransmitters of the serotonin and catecholamine systems for the first laboratory above are as follows:

Serotonin=175 to 225 micrograms of neurotransmitter per grain of creatinine.

Dopamine=125 to 175 micrograms of neurotransmitter per gram of creatinine.

Norepinephrine=30 to 55 micrograms of neurotransmitter per gram of creatinine.

Epinephrine=8 to 12 micrograms of neurotransmitter per gram of creatinine.

The optimal ranges for the neurotransmitters of the serotonin and catecholamine systems for the second laboratory above are as follows.

Serotonin=85.6 to 175.4 micrograms of neurotransmitter per gram of creatinine.

Dopamine=50.0 to 273.0 micrograms of neurotransmitter per gram of creatinine.

Norepinephrine=8.4 to 47.7 micrograms of neurotransmitter per gram of creatinine.

Epinephrine=3.2 to 14.8 micrograms of neurotransmitter per gram of creatinine.

THERAPEUTIC RANGES are the range to be obtained in treatment to insure that resolution of symptoms is affected without overloading the system on neurotransmitters. The therapeutic ranges of the neurotransmitters of the serotonin and catecholamine systems are as follows. It should be noted that these numbers are a relative guide in treatment and that the therapeutic range should not be fixed on the absolute numbers reported. These therapeutic ranges are independent of any laboratory variability. Instead, the therapeutic range is specific to the respective neurotransmitter dysfunction disease. In general, the therapeutic range for serotonin in neurotransmitter dysfunction is typically 800 to 2400 micrograms of neurotransmitter per gram of creatinine and in a phase three response. For example, the therapeutic range for serotonin in non-obesity neurotransmitter disease is reported at 800 to 1,200. A serotonin level of 1,600 or higher could be acceptable in some circumstances.

Serotonin=1,200 to 2,400 micrograms of neurotransmitter per gram of creatinine for treatment of obesity, obsessive compulsive disorder (COD), panic attacks and severe anxiety.

Serotonin=250 to 1,200 micrograms of neurotransmitter per gram of creatinine for disease not related to obesity. Such as in conditions that respond relatively early on in treatment such as migraine headaches and some chronic pain states.

In general, the therapeutic range for dopamine in neurotransmitter dysfunction is typically 300 to 600 micrograms of neurotransmitter per gram of creatinine.

The therapeutic range for dopamine in treatment of Parkinsonism is less than 20,000 micrograms of neurotransmitter per gram of creatinine, often in the 6,000 to 8,000 range, with treatment decisions driven by clinical outcomes.

The therapeutic range for dopamine for restless range syndrome is typically 1,500 to 2,000 micrograms of neurotransmitter per grain of creatinine.

In general, the therapeutic range for norepinephrine in neurotransmitter dysfunction is typically 7 to 65 micrograms of neurotransmitter per gram of creatinine.

In general, the therapeutic range for epinephrine in neurotransmitter dysfunction is typically 2 to 16 micrograms of neurotransmitter per gram of creatinine.

The goal of treatment is to establish neurotransmitter levels of the serotonin and catecholamine systems in the optimal range for subjects with no symptoms of neurotransmitter dysfunction and in the therapeutic range for subjects suffering from symptoms of neurotransmitter dysfunction.

### EXAMPLES

In order to facilitate a more complete understanding of the present invention, Examples are provided below. In a preferred embodiment, the dopa stabilization and optimization dosing begins after the serotonin levels are optimized. However, the scope of the invention is not limited to specific embodiments disclosed in these Examples, which are for purposes of illustration only.

### Example 1

As shown in Table 1 below, the subject of Example 1 was initially administered a dosing of dopa without any dopa precursor dosing. The subject's initial urinary laboratory assay had a dopamine level below the desired dopamine range. Subsequent increases in the dopa dosing resulted in dopamine neurotransmitter level fluctuation and levels outside of the desired dopamine range. On day 75, the dopa dosing was combined with a dopa precursor dosing (here tyrosine). The dopa precursor dosing combined with the dopa dosing resulted in more stabile urinary dopamine neurotransmitter levels. A relative increase in the dopa dosing when used in combination with the dopa precursor dosing resulted in more predictable and stabile laboratory assay results within the desired dopamine range of 300 to 600 milligrams of dopamine per gram of creatinine. The desired range of 300 to 600 milligrams of dopamine per gram of creatinine is independent of any variability attributable to the laboratory methodology. Also, the desired dopamine levels were achieved with a smaller dosing of dopa.

TABLE 1
Desired dopamine range = 300 to 600

| | Tyrosine | Dopa | |
|---|---|---|---|
| | dosing in | dosing in | Dopamine |
| day | mg | mg | level |
| 0 | 0 | 360 | 223 |
| 14 | 0 | 720 | 274 |
| 31 | 0 | 1,080 | 4,893 |
| 44 | 0 | 1,080 | 1,027 |
| 60 | 0 | 1,080 | 12,960 |
| 75 | 6,000 | 360 | 293 |
| 93 | 6,000 | 720 | 1,278 |
| 107 | 6,000 | 480 | 531 |
| 123 | 6,000 | 480 | 538 |
| 137 | 6,000 | 480 | 518 |
| 181 | 6,000 | 480 | 527 |

### Example 2

As shown in Table 2 below, the subject of Example 2 was initially administered a dosing of dopa precursor (here tyrosine) without a dosing of dopa. The subject's initial urinary laboratory assay had a dopamine level below the desired dopamine range. Subsequent increases in the dopa precusor dosing resulted in dopamine neurotransmitter level fluctuation and levels outside of the desired dopamine range. On day 91, the dopa precursor dosing was combined with a dopa dosing. The dopa precursor dosing combined with the dopa dosing resulted in more stabile urinary dopamine neurotransmitter levels. A relative increase in the dopa dosing when used in combination with the dopa precursor dosing resulted in more predictable and stabile laboratory assay results within the desired dopamine range of 300 to 600 milligrams of dopamine per gram of creatinine. The desired range of 300 to 600 milligrams of dopamine per gram of creatinine is independent of any variability attributable to the laboratory methodology. Also, the desired dopamine levels were achieved with a smaller dosing of the dopa precursor.

TABLE 2
Desired dopamine range = 300 to 600

| | Tyrosine | Dopa | |
|---|---|---|---|
| | dosing in | dosing in | Dopamine |
| Date | mg | mg | level |
| 0 | 6,000 | 0 | 164 |
| 12 | 7,5000 | 0 | 182 |
| 24 | 9,000 | 0 | 134 |
| 44 | 12,000 | 0 | 1,280 |
| 59 | 12,000 | 0 | 1,786 |
| 71 | 12,000 | 0 | 873 |
| 91 | 6,000 | 360 | 221 |
| 104 | 6,000 | 720 | 468 |
| 120 | 6,000 | 720 | 492 |
| 153 | 6,000 | 720 | 491 |

While the compositions and methods of the present invention have been described in terms of preferred embodiments, it will be apparent to those of skill in the art that variations may be applied to the compositions and methods and in the steps or in the sequence of steps of the method described herein without departing from the concept, spirit and scope of the present invention.

1. A method for optimizing control of the catecholamine system in a patient including: establishing a desired serotonin level in a patient; establishing an L-dopa precursor base including the step of administering an L-dopa precursor after establishing the serotonin level; and administering a direct source of dopamine to reach a desired stable dopamine level in the patient.

2. The method of claim 1 wherein the step of administering the L-dopa precursor includes the step of administering the precursor in a dosing range of about 750 mg to 9,000 mg per day.

3. The method of claim 1 wherein the step of administering the direct source of dopamine includes the step of administering the dopamine in a dosing range of about 12 mg to 4,800 mg per day.

4. The method of claim 3 wherein the direct source of dopamine is L-dopa.

5. The method claim 1 wherein the desired dopamine level is within a range of about 300 micrograms to 600 micrograms per gram of creatinine.

6. The method of claim 1 further including the step of repeatedly assaying serum of the patient to determine the stability of the patient's dopamine level.

7. The method of claim 6 wherein the step of assaying includes performing the assay on serum or fluid selected from the group consisting of central nervous system fluid, saliva, peripheral plasma, serum from blood and urine.

8. The method of claim 6 wherein the stable dopamine level varies less than 20 percent from a first assay to a second assay independent of laboratory variability.

9. A method for optimizing control of the catecholamine system in a patient suffering symptoms of neurotransmitter dysfunction including: establishing an L-dopa precursor base including the step of administering an L-dopa precursor in a dosing range of about 50 mg to 14,000 mg per day; and administering a direct source of dopamine to reach a desired stable dopamine level in the patient to alleviate the patient's symptoms of neurotransmitter dysfunction.

10. The method of claim 9 further including an initial step of establishing a desired serotonin level in the patient.

11. The method of claim 9 wherein the desired level of dopamine is in the range of about 300 micrograms to 600 micrograms per gram of creatinine.

12. The method of claim 9 further including the step of repeatedly assaying serum of the patient to determine the stability of the patient's dopamine level.

13. The method of claim 12 wherein the step of assaying includes performing the assay on serum or fluid selected from the group consisting of central nervous system fluid, saliva, peripheral plasma, serum from blood and urine.

14. The method of claim 12 wherein the stable dopamine level varies less than 20 percent from a first assay to a second assay independent of laboratory variability.
